(19) United States
(12) Patent Application Publication
(10) Pub. No.: US 2005/0089530 A1
(43) Pub. Date: Apr. 28, 2005

(54) Selenium yeast product, a method of preparing a selenium yeast product and the use of the product for preparing food, a dietary supplement or a drug

(75) Inventors: Sven Moesgaard, Almind (DK); Helge Skousen Paulin, Vamdrup (DK)

Correspondence Address: OBLON, SPIVAK, MCCLELLAND, MAIER & NEUSTADT, P.C., 1940 DUKE STREET, ALEXANDRIA, VA, 22314, US

(73) Assignee: PHARMA NORD APS, Vojens (DK)

(21) Appl. No.: 10/507,202

(22) PCT Filed: Mar. 14, 2003
(86) PCT No.: PCT/DK03/00167

(30) Foreign Application Priority Data
Mar. 15, 2002 (DK) PA 2002 00408

(51) Int. Cl. A61K 35/70; A61K 35/72; C12N 1/18

(52) U.S. Cl. 424/195.16; 435/254.2

(57) ABSTRACT

A selenium yeast product for use in food, dietary supplements or drugs containing significant and homogeneous amounts of easily digestible organically bound selenium, in which the content of selenium compounds is in the range of between 1000 and 1600 ppm, and in which product the content of 1-selenomethionine constantly constitutes at least 55% of the total selenium content, and in which product the content of selenium in inorganic selenium compounds does not exced 1% of the total selenium content. Furthermore, a method of preparing a selenium yeast product for use in food, dietary supplements, or drugs whereby said yeast is cultivated on a minimal medium under aerobic conditions, while nutrients are added to the yeast during the cultivation to an extent corresponding to the consumption of said nutrients in the yeast; glucose and/or maltose are the sole sources of carbon in the feeding medium.

### TECHNICAL FIELD

The present invention relates to a selenium yeast product for use in food, dietary supplements or drugs, said product containing significant and homogeneous amounts of easily digestible and tolerable, organically bound selenium. The invention also relates to a method of preparing said selenium yeast product as well as the use of the selenium yeast product for preparing food, a dietary supplement or a drug.

### BACKGROUND ART

Selenium is an element essential to human nutrition. Selenium is ingested through the diet, which, however, has a varying content of selenium. In large parts of the world, crops with poor levels of selenium are cultivated because the presence of said element in the soil is modest.

The importance of selenium to humans has been substantiated through a great number of tests. Selenium is incorporated into different organic molecules, including in particular amino acids. 1-selenomethionine, selenocysteine, selenocystine and selenocystine are the most important compounds. Thus, selenium is part of proteins, which are of structural importance to the body. Furthermore, selenium is an important ingredient in a number of enzymes which influence the metabolism, the reproduction, the prevention of cancer, the immnune defence and the psyche of humans. viz. Rayman, M., The importance of selenium to human health, Lancet 356:233-241 (2000).

Due to the often insufficient content of selenium in the ordinary diet, it is advantageous to add selenium in form of enrichment, dietary supplements or drugs. These may include an inorganic selenium such as selenite, or they may include organic sources, including selenium yeast. There is a significant difference between absorption and toxicity of inorganic and organic selenium, the inorganic compounds usually being absorbed significantly slower and also being more toxic than organic sources of selenium. An often used source of organic selenium is yeast with selenium content.

When cultivating yeast, it is possible to add the nutrient medium selenium in form of inorganic compounds, including sodium selenite and sodium selenate. The selenium added to the nutrient medium in this way is largely absorbed by yeast and incorporated into organic compounds, including 1-selenomethionine.

Selenium yeast may be prepared by use of a number of yeast species, including Saccharomyces charomnyces cerevisiae, Saccharornyces boulardii sequela and Saccharomyces torula, and by use of different cultivation conditions. As a result, a variable selection of organic compounds of selenium can be formed in the yeast. The reproducibility obtained by known methods can be unsatisfactory. A poor reproducibility has caused a reluctance to use selenium yeast by authorities, researchers and consumers. The Scientific Committee on Foods states that the 1-selenomethionine content in selenium yeast varies between 20 and 50%, viz European Commission, Scientific Committee on Food, Opinion on substances for nutritional purposes which have been proposed for use in the manufacture of foods for particular nutritional purposes, viz. “PARNUTS”, 12 May 1999, page 5. Scientific Committee on Food on the Tolerable Upper Intake Level of Selenium, 11 Oct. 2000, page 2. It is of vital importance to the use in dietary supplements or drugs that the essential compounds of selenium, viz. species, are present in a homogenous concentration. This allows long-term studies of the importance of selenium to be carried out where the reproducibility from production to production is of essential importance for interpreting and using the results from the studies.

As a cormnercial product, selenium yeast is generally prepared by cultivating on molasses which have varying compositions. It is known from literature that different types of selenium yeast cause varying absorption levels and thus deviating responses, viz. Clausen, J. et al., A comparison of ten selenium supplementation products, Selenium in Medicine and Biology, Walter de Gruyter & Co 305-14 (1988). A number of patents describe the cultivation of selenium yeast containing different concentrations of selenium Thus, U.S. Pat. No. 4,530,849 discloses the cultivation of selenium yeast with approx. 1000 ppm of selenium. However, the patent does not disclose how the selenium is bound in the yeast or to which extent the method can be reproduced.

Patent application WO 98/37172 relates also to the cultivation and the use of selenium yeast. The method by cultivation includes the steps of admixture of a water-soluble salt of selenium with a nutrient medium followed by the addition of an aqueous suspension of yeast. The resulting selenium yeast has a selenium content, which is not divided into constant, homogeneous compounds. The possibility of obtaining a homogeneous yield and composition of selenium compounds is not described either.

It is a known fact that yeast and other microorganisms can be cultivated on a medium containing a minimum level of nutrients, a so-called minimal medium. However, such a method is not usually used industrially because it might result in a poor yield and an undesired composition of the microorganisms. Thus, it is surprising that it is possible by cultivation on a minimal medium in accordance with the present invention to obtain a good yield and a reproducible composition of selenium yeast which is optimal in human nutrition.

Thus, the present invention relates to a method of preparing a yeast product containing significant and homogeneous amounts of digestible, organically bound selenium. The resulting yeast is a powder which may be used directly or compressed into tablets by conventional techniques. These tablets can be marketed as food, dietary supplements or drugs.

### BRIEF DESCRIPTION OF THE INVENTION

In a first aspect, the invention thus relates to a selenium yeast product for use in food, dietary supplements or drugs, said product being characterised by having a content of organic selenium compounds corresponding to between 1000 and 1600 ppm, preferably between 1100 ppm and 1500 ppm, most preferably between 1200 ppm and 1400 ppm of selenium, and by the content of 1-selenomethionine constantly constituting at least 55% of the total selenium content, and by the content of selenium in inorganic selenium compounds not exceeding 1% of the total selenium content.

The resulting product is furthermore characterised by a human absorption of more than 85%.

In a second aspect, the invention relates to a method of preparing a selenium yeast product for use in food, dietary supplements or drugs, whereby the yeast is cultivated under aerobic conditions, said method being characterised by
i) nutrients being fed to the yeast during the cultivation to an extent corresponding to the consumption of said nutrients in the yeast;
ii) glucose and/or maltose being the only sources of carbon in the feeding medium;
iii) the concentration of ethanol during the cultivation not exceeding 1%, preferably 0.5% and most preferably 0.2%;
iv) the pH value during the cultivation being maintained at between 4.0 and 6.0, preferably between 4.4 and 5.7, most preferably between 4.7 and 5.4, such as 5.0; and
v) the feeding medium being mixed with an aqueous salt of selenium in an amount corresponding to between 1000 and 1500 ppm of selenium, calculated on dry matter in the yeast.

In a third aspect, the invention relates to a selenium yeast product prepared by the method of the invention.

In a fourth aspect, the invention relates to food, a dietary supplement or a drug including a selenium yeast product according to the invention.

In a fifth aspect, the invention relates to the use of the selenium yeast product according to the invention for preparing food, a dietary supplement or a drug.

### BRIEF DESCRIPTION OF THE INVENTION

Prior to the detailed description of the different embodiments of the invention, a number of relevant definitions specific to the main aspects of the invention are provided below.

Definitions

Minimal medium: Medium containing the rninimum amount of nutrients necessary for obtaining yeast growth.

Pharmaceutical quality: Properties of products described in a national pharmacopoeia

Starting medium: Mixture of water and additional nutrients seeded with yeast

Feeding medium: nutrients added to the starting medium/culture subsequent to the pitching of yeast.

The Zak-method: Method of cultivating yeast, whereby said yeast is pitched to a starting medium followed by nutrients being added under aerobic conditions through a feeding medium. The addition of nutrients is carried out under aerobic conditions at a rate corresponding to the absorption rate of said nutrients in the yeast. A correct control results in a formation of only a small amount of alcohol. The alcohol formed is consumed by the yeast in the last stage of the cultivation. The method is conventional and is referred to as the Z-method. It is as a principle developed by Mr Søren Sak and described in Danish patent No. 28507 (1921).

Human absorption: Difference between ingested amount of isotope and excreted amount of isotope in defecation. Human absorption is calculated in percent of the ingested amount of isotope.

As mentioned above, hitherto known methods of preparing selenium yeast products use primarily molasses as a carbon source and when a glucose based medium is used, cf. WO 98/37172, this method deviates from the method of the invention by not ensuring a continuous addition of nutrients and selenium which corresponds to the cell growth. In addition, an admixture of selenium is carried out within a relatively short period of time, which complicates the formation of organic compounds of selenium, which have particular importance in the human nutrition.

The nutrients for the cultivation of yeast according to the invention include sources of carbon and nitrogen as well as micro nutrients in form of vitamins and minerals. The carbon must be in a form which can be ingested immediately during the cultivation and therefore it must be highly soluble in water and have a composition allowing a consumption by means of the enzymes present in the yeast. These carbon sources include glucose and maltose which can be purified into glucose syrup in such a manner that they can be used as nutrients for microorganisms producing food, dietary supplements or drugs. As a source of nitrogen, it is possible to use inorganic compounds, including ammonia with a sufficiently high purity level so as to avoid toxicity or formation of undesired compounds. The metabolism of the yeast is not completely known and consequently it is necessary to add micro nutrients in form of yeast extract, which is described in pharmacopoeia.

Thus, yeast can according to the invention be prepared on the basis of raw materials of a pharmaceutical quality and composed in such a manner that the yeast has a minimum level of nutrients which can be used for growth. A typical method of cultivating the yeast includes the following steps:
1) Processing the nutrient medium
2) Seeding with pitching yeast
3) Cultivating
4) Harvesting
5) Washing
6) Heat treating
7) Drying

The nutrient medium is produced by dissolving sugar substances, vitamins and minerals in water heated to between 24 and 37° C., preferably between 26 and 34° C., and best between 28 and 32° C. prior to the pitching of yeast.

A seeding with pitching yeast is carried out to the complete and heated nutrient medium by suspending the yeast cells in the medium while stirring at a frequency of between 1 and 2 Hz. The selenium yeast can be prepared by use of a number of species, including Saccharomyces cerevisiae, Saccharomyces boulardii sequela and Saccharomyces torula. Among said species, Saccharomyces cerevisiae is generally considered to be suitable for human ingestion, and it is widely used for the preparation of bread and alcohol. For the preparation of a selenium yeast product according to the invention it is an advantage to use a production strain, which is genetically stable and thus less inclined to be subjected to a mutation. In an embodiment according to the present invention, the strain ATCC No 74366 is thus used without, however, limiting the invention thereto.

The cultivation is carried out on the basis of a nutrient medium which is produced as a particular minimal medium and seeded with the above pitching yeast. Furthermore, additional carbohydrates are added in form of glucose and/or maltose. An aqueous solution of ammonia is admixed as a source of nitrogen. In order to ensure the highest possible growth and to counteract the formation of alcohol, large amounts of air are introduced, preferably atmospheric air containing sufficient oxygen. In large scale preparations, it is in practice difficult to ensure a complete oxidation, which can change the nutrition need of the yeast slightly. Cultivation is terrninated at a concentration of yeast of approximately 4% by weight, calculated on the total content of nutrient medium and yeast. The addition of nutrients is carried out under aerobic conditions at a rate corresponding to the absorption rate of said nutrients in the yeast. As a result, only a small amount alcohol is formed through a correct control. The alcohol formed is consumed by the yeast in the last stage of the cultivation.

In practice, the addition of nutrients in the correct amount is controlled by continuously measuring the amount of alcohol in form of ethanol formed during the growth. If the nutrients are added too slowly, no alcohol is formed and if the addition is carried out too fast, significant amounts of alcohols are formed. Therefore, the amount of alcohol present at any time should not exceed 1%, preferably 0.5% and most preferably 0.2%. Furthermore, the pH of the growth medium can be used as an indicator for maintaining the correct balance between consumption and addition of nutrients, and therefore the pH is controlled and adjusted during the growth so as to maintain the pH between 4.0 and 6.0, preferably between 4.4 and 5.7, most preferably between 4.7 and 5.4, such as 5.0.

The harvest involves a separation of the cultivated yeast from the nutrient medium. This separation is best achieved by centrifuging, whereby a concentrated yeast cream is produced containing approximately 20% by weight of yeast

The purpose of a washing is to remove the excess of nutrients in the yeast cream. The washing is most advantageously carried out by adding water followed by a centrifuging so that a yeast cream is produced. In this way, the yeast is washed 2 to 6 times and preferably 4 times, whereby practically all extracellular nutrients are removed from the yeast cream.

The purpose of the heat treatment is to kill the yeast cells so that the selenium present therein becomes available for the human digestion. A heat treatment results also in an extensive disintegration of the yeast cells. An advantageous heat treatment can be carried out as a pasteurization in a plate heat exchanger at a temperature of 87° C. and a standing time of 30 seconds.

The yeast can be dried by conventional methods of drying organic material including freeze drying, drum drying, tray drying or spray drying, preferably spray drying. A spray drying removes water at an input temperature for air of between 160 and 240° C., preferably between 180 and 220° C. and best about 200° C. The output temperature can in this connection be from 70 to 90° C., preferably 80 to 90° C. and best about 86° C. The resulting powder has a water content of between 4 and 9%, preferably between 6 and 9% and best about 8%. The powder can subsequently be treated by way of moistening with water and followed by a drying so as thereby to improve the capability to absorb water later on.

Selenium yeast according to the invention can be used for preparing food, dietary supplements or drugs, either as the only source of selenium or in combination with other selenium containing ingredients.

Thus, the invention also relates to food, a dietary supplement or a drug, which as a source of selenium uses the selenium yeast according to the invention.

In an embodiment of the invention, the use includes a product including a disintegrating agent, a flow agent as well as selenium yeast. This mixture is compressed into a tablet containing between 25 and 800 μg of selenium, particularly between 40 and 300 μg of selenium and especially 50 to 200 μg of selenium per tablet

However, in addition to the scope of application described above the invention can also be used for other types of food to be enriched with selenium such as flour, other powdery food as well as drinks.

The invention is explained in detail below with reference to the following examples.

### EXAMPLES

### Example 1

1 a)

In this example, a fermentor with a volume of 0.014 m³ is used.

At the beginning of the cultivation, a nutrient medium is prepared with the following composition of raw materials of a pharmaceutical quality:

| | | |
|---|---|---|
| Water | Ph. Eur. | 5,400 g |
| Glucose syrup | Ph. Eur. | 31.8 g |
| KH₂PO₄ | Ph. Eur. | 25 g |
| Ammonia water 2.5% | Ph. Eur. | 114 g |
| Biotin (0.01%) | Ph. Eur. | 2.1 ml |
| Thiamine hydrochloride (1.0%) | Ph. Eur. | 2.5 ml |
| Calcium pantothenate | Ph. Eur. | 0.08 g |
| Yeast extract | USP | 75 g |
| Iron sulphate | Ph. Eur | 0.10 g |
| Magnesium sulphate | Ph. Eur. | 5.0 g |
| Manganese sulphate | Ph. Eur. | 0.033 g |
| Zinc sulphate | Ph. Eur | 0.033 g |

Subsequent to adjusting the temperature at 30° C., 5 g of pitching yeast is admixed. The seeded nutrient medium is blown through with sterile atmospheric air in an amount of 20 litres per minute. A stirring is carried out by means of a spindle at a frequency of 17 Hz. Sulphuric acid is used to adjust the pH so as to maintain the pH at between 4.7 and 5.4. It is controlled that ethanol does not exceed 0.2%, and in addition the concentration of ethanol is maintained as close to 0 as possible.

During the cultivation, nutrients are added with the feeding medium in the following amounts:

| | | |
|---|---|---|
| Glucose syrup | Ph. Eur | 1,060.5 g |
| Ammonia water 2.5% | Ph. Eur. | 1,451 g |

In addition, sodium selenite is admixed in ammonia water 2.5%, as follows:

| | | |
|---|---|---|
| Sodium selenite | Ph. Eur | 1.2758 g |

Glucose syrup, ammonia water and sodium selenite are added at a rate corresponding to the consumption rate of the substances in the yeast. This is controlled by continuously measuring the formation of alcohol. The concentration of alcohol is maintained close to 0.

The addition of ammonia water (2.5%) is terminated after 18 hours. After 19 hours, the cultivation is terminated.

The harvest of the yeast is carried out by transferring the medium with the yeast to a centrifuge wherein the yeast cream is separated from the excess medium within 5 minutes. The resulting yeast cream with a dry matter content of approx. 20% is admixed 5,000 g of water. Subsequently, the yeast mixture is centrifuged again. The washing water is removed and the resulting yeast cream is admixed 5,000 g of water. The process is repeated 4 times to separate the yeast cells from the medium.

The washed yeast cream is carried through a plate pasteurizer in which it is subjected to a heat treatment at 87° C. with a standing time of 30 seconds. Immediately after the heat treatment, the yeast cream is cooled to 4° C. in a plate pasteurizer. The amount of yeast cream is 1,897.5 g.

The yeast cream is transferred to a freeze dryer. Subsequent to freezing at −40° C. for 24 hours, the water sublimes within 18 hours. 380 g of dried selenium yeast is hereby provided having a water content of 0.2% and a concentration of selenium of 1,380 ppm in dry matter. The dietary properties and the reproducibility of the resulting selenium yeast product prepared are determined in the manner described below in Examples 2, 4 and 5.

1 b)

Yeast is prepared in accordance with Example 1a above. However, a fermentor with a volume of 150 m³ is used. The nutrient medium is composed of corresponding ingredients in the same mutual proportions and has a total weight of 56,848 kg.

Glucose syrup and ammonia water 2.5% are added in the same way as in Example 1 a. For this purpose, 13,500 kg of glucose syrup and 1,410.5 kg of ammonia water 25% are used. Selenium in form of sodium selenite is admixed in an amount of 15 kg. The addition of ammonia water is terminated after 18 hours and the cultivation is terminated after 19 hours.

The washing and the heat treatment of the yeast are carried out according to the principles described in Example 1a.

The drying of the yeast is carried out by spray drying in a drying tower having a rotating sprayer wheel. The frequency of the sprayer wheel is set at 167 Hz The air temperature for drying is set at 200° C. The resulting starting temperature is 86° C. The total amount of powder with a water content of 7% is 5,035 kg. The concentration of selenium is 1,355 ppm on dry matter. The dietary properties and the reproducibility of the resulting selenium yeast product prepared are determined in the manner described below in Examples 2, 4 and 5.

### Example 2

Digestibility and absorption of selenium yeast.

2 a) In Vitro Digestibility of Selenium

As a basis for determining the in vitro digestibility of selenium yeast, a further development of the method 9.1 of 15 Nov. 1994 of The Danish Plant Directorate for determining the enzyme-digestible organic matter in pigs, viz. EFOS pigs, is used. The method has been changed so as to correspond to the conditions of the human digestion. Enzymes, which can decompose cellulose, are thus not included in this in vitro study.

The principle is treatment with pepsin followed by treatment with pancreatin. Undisolved sample material is filtered off and dried. By comparing the determinations of selenium in the original sample with the content in the filtrate and the retentate, the digestibility of selenium is calculated. Thus, selenium yeast is mixed at pH 2.0 with pepsin and incubated at 40° C. for 75 minutes followed by treatment with pancreatin at pH 6.8 at 40° C. for 3 hours and 30 minutes. Subsequently, a filtration is carried out by means of vacuum and the selenium content in the filtrate and the retentate, respectively, is determined. The selenium content in the filtrate in percent of the total selenium content in the sample is an expression of the in vitro digestibility of selenium yeast.

A selenium yeast is obtained with the following characteristics:

Digestible selenium: 99%

2 b) In Vivo Absorption and Retention of Selenium

As a basis for determining the in vivo digestibility, selenium yeast according to the invention is administered where a stable isotope having a content of selenium-77 of 99.3% is used for the cultivation. Since naturally occurring selenium only contains 7.8% Se-77 and at the same time contains 49.82% of Se-80, it is possible to determine the proportion in human material originating from such an addition of isotope by measuring these isotopes via ICP-MS, viz. Inductively Coupled Plasma—Mass Spectrometry.

The determination of in vivo absorption, retention and bioaccessibility is moreover carried out as follows:

Twelve male participants aged 20 to 55 are administered a single dose of selenium yeast corresponding to 300 μg selenium-77. The participants collect faeces and urine for a period of 5 days, and 11 blood samples are collected during said period. In order to control the collection of faeces and urine, PABA, viz. para-amnino-benzoic-acid, and plastic tubes, respectively, are administered which can be recovered in both the urine and the faeces as a control of the collection. The absorbed amount of selenium-77 is determined by comparing the ingestion of selenium-77 with the extraction through faeces.

The retention, viz. the retained amount, is determined as administered trace amount minus excretion through faeces and urine compared to the administered trace amount The blood samples are used to describe the pharmaco-dynamics.

The following characteristics for absorption and retention of the selenium yeast according to the invention are obtained:

The absorption is 89%. The retention is 74%.

2 c) In Vivo Dosage/Response at Long-Term Ingestion

A blank experiment was carried out for a continuous period of 2 years. A group of 49 persons ingested a tablet each day with a content of 0, 100, 200 or 300 μg of selenium in form of selenium yeast according to the invention. Furthermore, the participants ingested through their food the amount of selenium naturally occurring in the diet, viz. approximately 50 μg/day. After 2 years where a balance between absorption and excretion has been obtained, the selenium content in whole blood was determined.

The following results were found:

| | | Mean value of | | Increase |
|---|---|---|---|---|
| Ingestion per | | selenium in | Absorption of | compared |
| day | Number of | whole blood | selenium per | to placebo |
| (μg) | participants | (μg/l) | μg (μg/l/μg) | (%) |
| 0 | 17 | 95.6 | — | 0 |
| 100 | 11 | 177.2 | 0.816 | 85 |
| 200 | 8 | 307.6 | 1.06 | 222 |
| 300 | 13 | 440.8 | 1.15 | 361 |

Based on the above results, a linear connection between the ingested dosage of selenium yeast and selenium content in whole blood could be determined and expressed in the following way:
Selenium in whole blood (μg/l)=1.12 ×ingestion (μg/day)+38

The discovered response on selenium yeast according to the invention surpasses the prior art description of selenium yeast, viz. Schrauzer, G. N., Selenium in human nutrition, Bioinorganic Chemistry, 8:303-318 (1978).

2 d) Measurement of Side Effects at Long-Term Ingestion

For a period of 1-2½ years, selenium yeast according to the invention was administered in doses of 100, 200 or 300 μg or placebo to 806 persons in 4 equally divided groups. The period covers approximately 1400 person-years. During this period, the tolerance and the side effects were tested in all persons, 2.6%, viz. 21 of the 806 persons, reported side effects. Before the randomization was terminated, the side effects were categorised as either mild, 17 persons, moderate, 4 persons, or serious, 0 persons.

Subsequent to divulgating the randomization, the side effects turned out to be divided randomly on the groups, 8 relating to placebo, 8 to 200 μg Se and 6 to 300 μg Se. The conclusion was that the side effects were insignificant and did not relate to the selenium yeast according to the invention.

### Example 3

Tablet:

Preparation of tablet of selenium yeast according to the invention is carried out by means of the following ingredients:

Selenium yeast according to the invention

Tablet auxiliaries: microcrystalline cellulose
silicon dioxide
magnesium salts of fatty acids

Flow agent: di-calcium phosphate

Surface-treatment agent: hydroxyl propyl methyl cellulose
talcum

Colourant: titanium dioxide

The ingredients are mixed in a conventional manner and compressed into tablets so that each tablet contains 100 μg of selenium originating from selenium yeast.

### Example 4

Speciation of selenium yeast

A selenium yeast cultivated in a conventional manner by use of molasses and 2 batches of selenium yeast according to the invention were examined for content of various compounds of selenium. The samples were subjected to acid hydrolysis with thioglycolic acid as stabilizer in oxygen-free surroundings. The content of selenomethionine and sodium selenite was then determined by HPLC against laboratory references.

The selenium yeast cultivated on molasses had a content of 49% of selenomethionine of the extractable and HPLC-accessible part, while the selenium yeast according to the invention in two independent production batches showed a content of 72.8 and 72.9%, respectively, of selenomethionine of the extractable and HPLC-accessible part.

The extractable part of total selenium exists as 95 to 101%, of which approximately 80% can be tested by means of HPLC. The minimum true amount of 1-seleno-methionine of total selenium is thus 72.8%×95%×80%=55.3%. The highest possible true amount is approx. 90%. The measured amount of sodium selenite is less than 1%. It is possible to use a method described by Erik. H. Larsen et al., viz. Larsen, E. H. et al., J. Anal. At. Spectrom., 16, 1403-1408, 2000, for determining species of selenium.

In the Example, importance is attached to the reproducibility rather than to the actual true amount.

### Example 5

In vivo absorption of selenium yeast and inorganic selenium

In the same way as in the application Example 2b, either selenium yeast according to the invention or inorganic selenium-77 was administered to the same 12 test persons. The human absorption of the two sources of selenium was 89% for selenium yeast according to the invention and 23% for inorganic seleniurn, respectively.

1. A method of preparing a selenium yeast product for use in food, dietary supplements, or drugs, whereby said yeast is cultivated on a minimal medium under aerobic conditions, comprising the steps of
a) cultivating the yeast, which includes
i) nutrients being fed to the yeast during the cultivation to an extent corresponding to the consumption of said nutrients in the yeast;
ii) glucose and/or maltose being the sole sources of carbon in the feeding medium;
iii) the concentration of ethanol during the cultivation not exceeding 1%, preferably 0.5% and most preferably 0.2%;
iv) the pH value during the cultivation being maintained at between 4.0 and 6.0, preferably between 4.4 and 5.7, most preferably between 4.7 and 5.4, such as 5.0; and
v) an aqueous salt of selenium being admixed to the feeding medium in an amount corresponding to between 1000 and 1500 ppm of selenium, calculated on dry matter in the yeast;
b) isolating the yeast obtained in step (a).

2. A method according to claim 1, further comprising the isolation including harvest by way of centrifuging or filtration.

3. A method according to claim 1, further comprising including the steps of:
c) washing the yeast cells from step (b),
d) heat treating the yeast cells from step (c), and
e) optionally drying the product from step (d).

4. A method according to claim 1 further comprising the minimal medium being composed of raw materials of a pharmaceutical quality.

5. A method according to claim 1 further comprising the yeast including a species of the genus Saccharomycetaceae, preferably Saccharomyces cerevisiae, Saccharomyces boulardii sequela and/or Saccharomyces torula.

6. A method according to claim 5, wherein the yeast is Saccharomyces cerevisiae.

7. A selenium yeast product for use in food, dietary supplements or drugs, comprising
a) a content of organic selenium compounds corresponding to a range of between 1000 and 1600 ppm of selenium, preferably between 1100 ppm and 1500 ppm of selenium, most preferably between 1200 ppm and 1400 ppm of selenium,
b) the content of 1-selenomethionine constantly constituting at least 55% of the total selenium content, and the content of selenium in inorganic selenium compounds not exceeding 1% of the total selenium content,
c) the selenium yeast product being obtainable by cultivating a yeast culture seeded with a pure culture of a Saccharomyces sp., preferably S. cerevisiae, S. boulardii sequela, and/or S. torula, by adding sources of carbon, nitrogen and selenium in amounts per time unit corresponding to the amount which can be absorbed in the yeast during a predetermined time period, and the cultivation taking place in minimal medium exclusively including purified, homogeneously defined nutrients in form of raw materials which are described in pharmacopoeia.

8. A selenium yeast product according to claim 7, comprising producing said selenium yeast product by the method according to claim 1.

9. A method for preparing a food product comprising adding said selenium yeast product according to claim 7 to said food product.

10. A method for preparing a dietary supplement comprising adding said selenium yeast product according to claim 7 to said dietary supplement.

11. A method for preparing a drug comprising adding said selenium yeast product according to claim 7 to said drug.
